(19) United States
(12) Patent Application Publication
(10) Pub. No.: US 2011/0054455 A1
(43) Pub. Date: Mar. 3, 2011

(54) Electromagnetic Thermotherapeutic Apparatus

(76) Inventors: Gwo-Bin Lee, Tainan City (TW); Xi-Zhang Lin, Tainan City (TW); Sheng-Chieh Huang, Tainan City (TW); Yi-Yuan Chang, Pingtung County (TW)

(21) Appl. No.: 12/835,206

(22) Filed: Jul. 13, 2010

(30) Foreign Application Priority Data
Aug. 26, 2009 (TW) 098128682

(51) Int. Cl. A61B 18/04

(52) U.S. Cl. 606/28

(57) ABSTRACT

An electromagnetic thermotherapeutic apparatus includes a tubular needle and an inner needle. The tubular needle has an electromagnetic inductive portion that is made from a material capable of generating heat when subjected to an induction magnetic field, and that has a hollow tip, and a non-electromagnetic inductive portion that is connected to the electromagnetic inductive portion oppositely of the hollow tip. The inner needle is removably insertable into the tubular needle from the non-electromagnetic inductive portion to the hollow tip.

### CROSS-REFERENCE TO RELATED APPLICATION

This application claims priority of Taiwanese application no. 098128682, filed on Aug. 26, 2009.

### BACKGROUND OF THE INVENTION

1. Field of the Invention

This invention relates to a therapeutic apparatus, more particularly to an electromagnetic thermotherapeutic apparatus.

2. Description of the Related Art

Generally, conventional methods of treating organ swelling (e.g., spleen swelling) and organ tumors include surgical resection, partial organ artery embolization (e.g., partial splenic artery embolization), radiofrequency ablation (RFA), radiation therapy, etc. The methods and drawbacks thereof are described as follow.

An incision of approximately 15-20 cm is required to be made at left upper abdomen for the surgical resection, thereby resulting in a long scar after the surgical resection. During the surgical resection, hemorrhage occurs and consequently increases danger to a patient. Intestinal adhesion may happen after the surgical resection. Furthermore, the surgical resection is relatively complicated.

Partial splenic artery embolization is an example of partial organ artery embolization and is conducted using the following steps. An incision of 0.5 cm is made at right inguen. A guide wire and a catheter are placed into a femoral artery and are delivered to a desired splenic artery. Embolic gel particles are subsequently injected so as to occlude a desired branch of the splenic artery. Even though partial splenic artery embolization can be performed by virtue of a minimally invasive surgery and is able to avoid the risk of surgical resection, the same is limited by patients' primary disease.

RFA employs a high temperature to induce coagulative necrosis of a desired tissue. An electric current is applied to a needle electrode so as to convert electric energy to heat energy. Accordingly, the high temperature can be generated. However, studies and literatures have indicated that general anesthetization is normally required for a patient during operation of RFA so as to prevent the patient from suffering enormous pain. Moreover, RFA is costly.

Radiation therapy involves using radiation to irradiate a desired portion and may give rise to many side effects.

### SUMMARY OF THE INVENTION

Therefore, the object of the present invention is to provide an electromagnetic thermotherapeutic apparatus that can overcome the aforesaid drawbacks of the prior art.

According to this invention, an electromagnetic thermotherapeutic apparatus includes a tubular needle and a first inner needle. The tubular needle has an electromagnetic inductive portion that is made from a material capable of generating heat when subjected to an induction magnetic field, and that has a hollow tip, and a non-electromagnetic inductive portion that is connected to the electromagnetic inductive portion oppositely of the hollow tip. The first inner needle is removably insertable into the tubular needle from the non-electromagnetic inductive portion to the hollow tip.

### BRIEF DESCRIPTION OF THE DRAWINGS

Other features and advantages of the present invention will become apparent in the following detailed description of the preferred embodiment of this invention, with reference to the accompanying drawings, in which:

FIG. 1 is a perspective view of the preferred embodiment of an electromagnetic thermotherapeutic apparatus according to this invention;

FIG. 2 is a schematic view to illustrate the electromagnetic thermotherapeutic apparatus of FIG. 1 in a state of use;

FIG. 3 is a fragmentary sectional view illustrating that a first inner needle is inserted in a tubular needle of the electromagnetic thermotherapeutic apparatus of FIG. 1; and

FIG. 4 is a fragmentary sectional view illustrating that a second inner needle is inserted in the tubular needle of the electromagnetic thermotherapeutic apparatus of FIG. 1.

### DETAILED DESCRIPTION OF THE PREFERRED EMBODIMENT

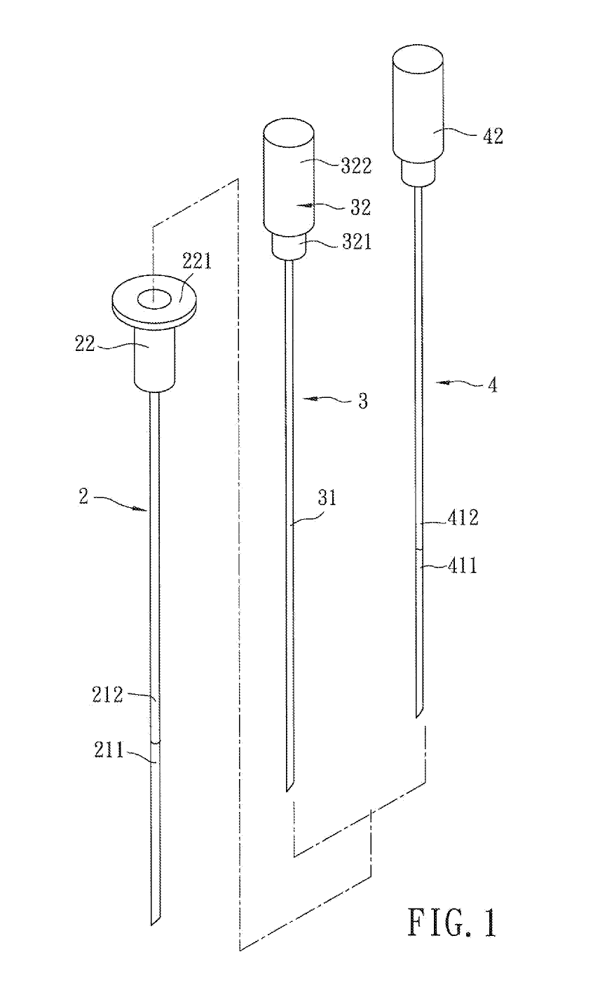

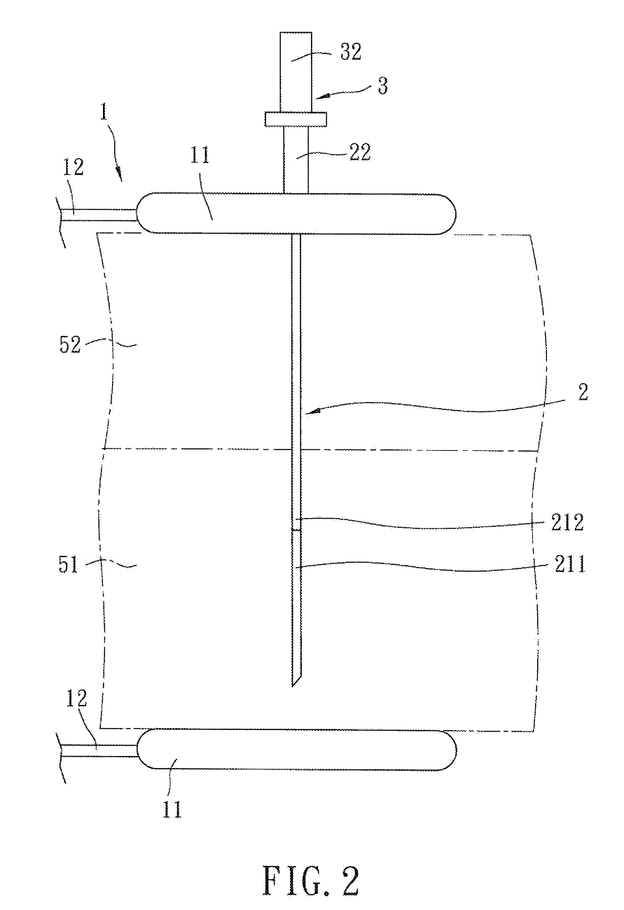

Referring to FIGS. 1 and 2, the preferred embodiment of an electromagnetic thermotherapeutic apparatus according to the present invention is able to generate heat when used with an electromagnetic induction heating device 1 that can produce a varying magnetic field, thereby being suitable to treat organ swelling (e.g., spleen swelling), organ tumors, etc. The electromagnetic induction heating device 1 includes two induction coils 11 that are spaced apart, and a connecting member 12 that connects the induction coils 11. When an alternating current is applied to the induction coils 11 through the connecting member 12, a varying high-frequency magnetic field is generated between the induction coils 11. It should be noted that other types of electromagnetic induction heating devices could also be used with the electromagnetic thermotherapeutic apparatus of this invention.

Referring to FIGS. 1, 3, and 4, the electromagnetic thermotherapeutic apparatus includes a tubular needle 2, a first inner needle 3, and a second inner needle 4. The tubular needle 2 has an electromagnetic inductive portion 211 that is made from a material capable of generating heat when subjected to an induction magnetic field, and that has a hollow tip 213, and a non-electromagnetic inductive portion 212 that is connected to the electromagnetic inductive portion 211 oppositely of the hollow tip 213. The hollow tip 213 of the electromagnetic inductive portion 211 forms an injection hole 214 adapted for injection. The tubular needle 2 further has an enlarged hollow head 22 that is connected to the non-electromagnetic inductive portion 212 oppositely of the electromagnetic inductive portion 211, and that has an end flange 221 projecting radially from one end of the enlarged hollow head 22. In this embodiment, each of the electromagnetic inductive portion 211 and the non-electromagnetic inductive portion 212 has an internal diameter of 0.8 mm and an external diameter of 1.47 mm.

The electromagnetic inductive portion 211 is made from a material which is able to generate heat when subjected to a varying magnetic field. In particular, the electromagnetic inductive portion 211 can be made from a metallic material since when the metallic material is subjected to a varying magnetic field, an eddy current is produced, thereby generating heat. If the electromagnetic inductive portion 211 is made from a ferromagnetic material (such as iron, cobalt, nickel, etc.) and is subjected to a varying magnetic field, in addition to an eddy current, the electromagnetic inductive portion 211 also generates heat due to magnetic hysteresis of the ferromagnetic material. In this embodiment, the electromagnetic inductive portion 211 is made from stainless steel.

The non-electromagnetic inductive portion 212 is made from a material incapable of generating heat when subjected to an induction magnetic field, and is preferably made from a nonmetallic material. In this embodiment, the non-electromagnetic inductive portion 212 is made from ceramic.

The first inner needle 3 is removably insertable into the tubular needle 2 from the enlarged hollow head 22 to the hollow tip 213, and has a core needle 31 and an enlarged inner head 32. The core needle 31 is solid and has a diameter of 0.58 mm. The enlarged inner head 32 is connected to an end of the core needle 31 opposite to a tip of the core needle 31, is stepped, and has a thin section 321 insertable into the enlarged hollow head 22 and a thick section 322 to extend out of the enlarged hollow head 22. The enlarged hollow head 22 and the enlarged inner head 32 cooperatively limit an insertion depth of the core needle 31 in the tubular needle 2. The enlarged inner head 32 is suitable for holding, and can facilitate pulling the first inner needle 3 out of the tubular needle 2. In this embodiment, the core needle 31 is a one piece needle, and is made from a nonmetallic material such that the core needle 31 is unable to generate heat when subjected to an induction magnetic field.

The second inner needle 4 is removably insertable into the tubular needle 2, and has an electromagnetic inductive section 411 and a non-electromagnetic inductive section 412. The electromagnetic inductive section 411 is solid, is to be inserted into the electromagnetic inductive portion 211 of the tubular needle 2, and is made from a material capable of generating heat when subjected to an induction magnetic field. The non-electromagnetic inductive section 412 is solid, is connected to the electromagnetic inductive section 411 oppositely of a tip of the electromagnetic inductive section 411, and is to be inserted into the non-electromagnetic inductive portion 212 of the tubular needle 2. Similarly, the electromagnetic inductive section 411 can be made from a metallic material. In this embodiment, the electromagnetic inductive section 411 is made from stainless steel. The non-electromagnetic inductive section 412 is made from a nonmetallic material. In this embodiment, the non-electromagnetic inductive section 412 is made from ceramic. In this embodiment, each of the electromagnetic inductive section 411 and the non-electromagnetic inductive section 412 has a diameter of 0.79 mm.

The second inner needle 4 further has an enlarged inner head 42 that is the same as the enlarged inner head 32 of the first inner needle 3 in terms of the structure and the function, and that is connected to the non-electromagnetic inductive section 412 oppositely of the electromagnetic inductive section 411.

Costs of the tubular needle 2, and the first and second inner needles 3, 4 are low. Furthermore, the tubular needle 2, and the first and second inner needles 3, 4 can be made disposable, thereby increasing safety of the electromagnetic thermotherapeutic apparatus of this invention.

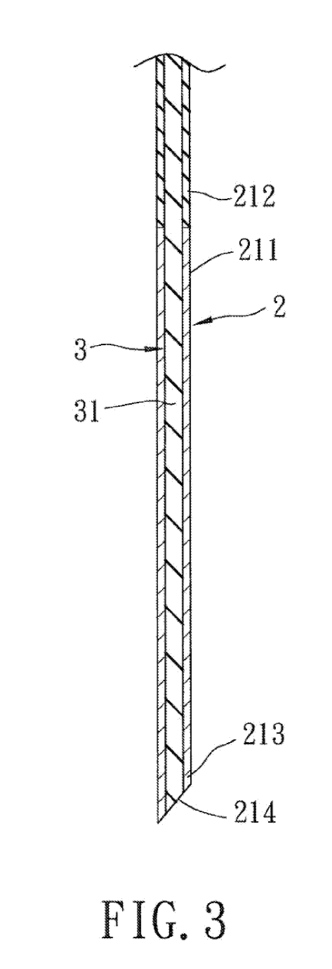

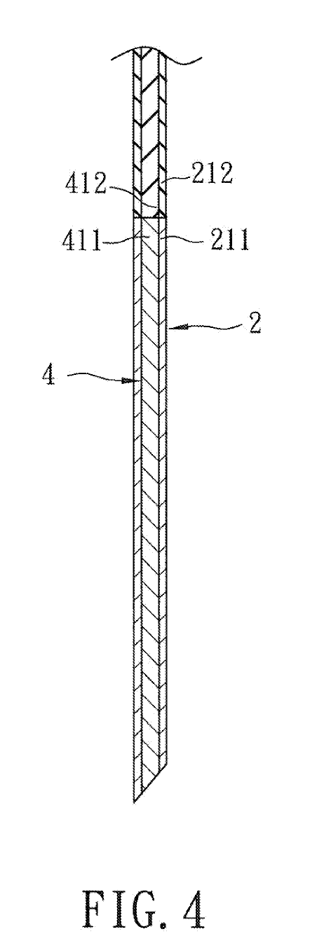

A method of using the electromagnetic thermotherapeutic apparatus is described as follows. First, the first inner needle 3 is inserted into the tubular needle 2 (see FIG. 3). Subsequently, the assembly of the tubular needle 2 and the first inner needle 3 is inserted into a treatment portion 51 so that the electromagnetic inductive portion 211 of the tubular needle 2 is disposed in contact with the treatment portion 51 (see FIG. 2). The first inner needle 3 is then pulled out of the tubular needle 2. The second inner needle 4 is inserted into the tubular needle 2 (see FIG. 4). The electromagnetic induction heating device 1 is placed at a proper position relative to the treatment portion 51 so that the treatment portion 51 is located between the two induction coils 11. Afterward, an electric current is applied to the electromagnetic induction heating device 1 such that the induction coils 11 produce a varying magnetic field. The electromagnetic inductive portion 211 of the tubular needle 2 and the electromagnetic inductive section 411 of the second inner needle 4 are simultaneously subjected to the varying magnetic field so that an eddy current is generated, thereby being able to produce heat and to cauterize the treatment portion 51.

It should be noted that the assembly of the tubular needle 2 and the first inner needle 3 have to penetrate a non-treatment portion 52 (e.g., epidermis, hypodermis, etc.) so as to reach the treatment portion 51 such as an organ (see FIG. 2).

When the electromagnetic thermotherapeutic apparatus is used to treat a rabbit with a swollen spleen, the aforementioned method is conducted. Before using the electromagnetic thermotherapeutic apparatus, the rabbit is anesthetized and is immobilized on an operating table, and the location of the spleen of the rabbit is detected using an ultrasonic scanner. The assembly of the tubular needle 2 and the first inner needle 3 is then inserted into a swollen portion of the spleen of the rabbit. The first inner needle 3 is pulled out of the tubular needle 2, and the second inner needle 4 is subsequently inserted into the tubular needle 2. The rabbit is subjected to a high-frequency magnetic field for several minutes so that the electromagnetic inductive portion 211 of the tubular needle 2 and the electromagnetic inductive section 411 of the second inner needle 4 continuously generate heat to cauterize the swollen portion of the spleen of the rabbit.

By virtue of the needle structures of the electromagnetic inductive portion 211 and the electromagnetic inductive section 411, the treatment portion 51 can be precisely cauterized. Furthermore, since only the non-electromagnetic inductive portion 212 is able to contact the non-treatment portion 52 during treatment, and since the non-electromagnetic inductive section 412 is disposed in the non-electromagnetic inductive portion 212, the healthy non-treatment portion 52 can be prevented from being cauterized (i.e., only the treatment portion 52 can be cauterized). Consequently, an effect of a treatment using the electromagnetic thermotherapeutic apparatus of this invention is satisfactory, and the treatment does not cause enormous pain. The electromagnetic thermotherapeutic apparatus of this invention is easy to operate and only results in small wound.

Medicine can be injected into a patient through the injection hole 214 of the tubular needle 2 so as to assist a treatment using the electromagnetic thermotherapeutic apparatus of this invention. However, it should be noted that since medicine is optional for the treatment using the electromagnetic thermotherapeutic apparatus of this invention, the injection hole 214 is not a necessary element for the tubular needle 2.

The core needle 31 of the first inner needle 3 is able to strengthen the tubular needle 2 by dint of the solid one-piece structure thereof when the assembly of the tubular needle 2 and the first inner needle 3 is inserted into the treatment portion 51, thereby facilitating the insertion of the tubular needle 2.

Even though the electromagnetic inductive section 411 of the second inner needle 4 is able to enhance heating and cauterizing effects of the electromagnetic thermotherapeutic apparatus, the second inner needle 4 is considered optional since the tubular needle 2 is capable of cauterizing the treatment portion 51 when used alone.

While the present invention has been described in connection with what is considered the most practical and preferred embodiment, it is understood that this invention is not limited to the disclosed embodiment but is intended to cover various arrangements included within the spirit and scope of the broadest interpretation and equivalent arrangements.

What is claimed is:

1. An electromagnetic thermotherapeutic apparatus comprising:
a tubular needle having an electromagnetic inductive portion that is made from a material capable of generating heat when subjected to an induction magnetic field, and that has a hollow tip, and a non-electromagnetic inductive portion that is connected to said electromagnetic inductive portion oppositely of said hollow tip; and
a first inner needle removably insertable into said tubular needle from said non-electromagnetic inductive portion to said hollow tip.

2. The electromagnetic thermotherapeutic apparatus of claim 1, wherein said first inner needle has a core needle that is a one-piece needle.

3. The electromagnetic thermotherapeutic apparatus of claim 1, further comprising a second inner needle removably insertable into said tubular needle, and having an electromagnetic inductive section that is to be inserted into said electromagnetic inductive portion of said tubular needle and that is made from a material capable of generating heat when subjected to an induction magnetic field, and a non-electromagnetic inductive section that is connected to said electromagnetic inductive section, and that is to be inserted into said non-electromagnetic inductive portion of said tubular needle.

4. The electromagnetic thermotherapeutic apparatus of claim 1, wherein said hollow tip of said electromagnetic inductive portion of said tubular needle forms an injection hole adapted for injection.

5. The electromagnetic thermotherapeutic apparatus of claim 3, wherein said electromagnetic inductive portion of said tubular needle and said electromagnetic inductive section of said second inner needle are made from a metallic material, and said non-electromagnetic inductive portion of said tubular needle and said non-electromagnetic inductive section of said second inner needle are made from a nonmetallic material.

6. The electromagnetic thermotherapeutic apparatus of claim 2, wherein said core needle of said first inner needle is made from a nonmetallic material.

7. The electromagnetic thermotherapeutic apparatus of claim 3, wherein said tubular needle further has an enlarged hollow head connected to said non-electromagnetic inductive portion oppositely of said electromagnetic inductive portion, and each of said first and second inner needles further has an enlarged inner head that is stepped and that has a thin section insertable into said enlarged hollow head, and a thick section to extend out of said enlarged hollow head.

8. The electromagnetic thermotherapeutic apparatus of claim 7, wherein said enlarged hollow head of said tubular needle has an end flange that projects radially from one end of said enlarged hollow head.
